(19) United States
(12) Patent Application Publication
(10) Pub. No.: US 2012/0295320 A1
(43) Pub. Date: Nov. 22, 2012

(54) APPARATUS AND METHOD FOR TREATMENT OF MICROORGANISMS DURING PROPAGATION, CONDITIONING AND FERMENTATION USING STABILIZED CHLORINE DIOXIDE/SODIUM CHLORITE WITH HOPS ACID EXTRACTS

(75) Inventor: Allen Ziegler, Los Angeles, CA (US)

(73) Assignee: RESONANT BIOSCIENCES, LLC, Palatine, IL (US)

(21) Appl. No.: 13/522,006

(22) PCT Filed: Jan. 14, 2011
(86) PCT No.: PCT/US11/21319

§ 371 Date: Jul. 12, 2012

Related U.S. Application Data
(60) Provisional application No. 61/295,627, filed on Jan. 15, 2010.

(51) Int. Cl. C12P 7/06; C12M 1/00

(52) U.S. Cl. 435/161; 435/289.1

(57) ABSTRACT

A method of reducing undesirable microorganism concentration, promoting desirable microorganism propagation/conditioning, and increasing desirable microorganism efficiency in an aqueous fluid stream includes (a) introducing a quantity of fermentable carbohydrate, sugar or cellulose to an aqueous fluid stream, (b) introducing a quantity of desirable microorganism to the aqueous fluid stream, (c) introducing a stabilized sodium chlorite solution into the aqueous fluid stream and (d) introducing a hops acid extract into said aqueous fluid stream. An apparatus for the same comprising a stabilized sodium chlorite batch tank, a hops acid extract tank and a process vessel wherein introducing stabilized sodium chlorite and hops acid extract solution from the batch tank and the hops acid extract tank to the process vessel promotes propagation of producing microorganisms present in the vessel.

### FIELD OF THE INVENTION

The present technology relates generally to anaerobic and aerobic microbial propagation, conditioning and/or fermentation. In particular, the present technology involves a method of reducing the concentration of undesirable microorganisms while simultaneously encouraging propagation and/or conditioning of desirable microorganisms and increasing the efficiency of desirable microorganisms during fermentation.

### BACKGROUND OF THE INVENTION

Microorganisms, such as yeast, fungi and bacteria, are used to produce a number of fermentation products, such as industrial grade ethanol, distilled spirits, beer, wine, pharmaceuticals and nutraceuticals (foodstuff that provides health benefits, such as fortified foods and dietary supplements). Yeast are also commonly utilized in the baking industry.

Yeast are the most commonly used microorganism in fermentation processes. Yeast are minute, often unicellular, fungi. They usually reproduce by budding or fission. One common type of yeast is Saccharomyces cerevisia, the species predominantly used in baking and fermentation. Non-Sacharomyces yeasts, also known as non-conventional yeasts, are also used to make a number of commercial products. Some examples of non-conventional yeasts include Kuyberomyces lactis, Yarrowia lipolytica, Hansenula polymorpha and Pichia pastoris.

However, other microorganisms can also be useful in making fermentation products. For example, cellulosic ethanol production, production of ethanol from cellulosic biomass, utilizes fungi and bacteria. Examples of these cellulolytic fungi include Trichoderma reesei and Trichoderma viride. One example of a bacteria used in cellulosic ethanol production is Clostridium Ijungdahlii.

Most of the yeast used in distilleries and fuel ethanol plants are purchased from manufacturers of specialty yeasts. The yeast are manufactured through a propagation process. Propagation involves growing a large quantity of yeast from a small lab culture of yeast. During propagation, the yeast are provided with the oxygen, nitrogen, sugars, proteins, lipids and ions that are necessary or desirable for optimal growth through aerobic respiration.

Once at the distillery, the yeast can undergo conditioning. The objective of both propagation and conditioning is to deliver a large volume of yeast to the fermentation tank with high viability, high budding and a low level of infection by other microorganisms. However, conditioning is unlike propagation in that it does not involve growing a large quantity from a small lab culture. During conditioning, conditions are provided to re-hydrate the yeast, bring them out of hibernation and allow for maximum anaerobic growth and reproduction.

Following propagation or conditioning, the yeast enter the fermentation process. The yeast are combined in an aqueous solution with fermentable sugars. The yeast consume the sugars, converting them into aliphatic alcohols, such as ethanol.

During these three processes the yeast can become contaminated with bacteria or other undesirable microorganisms. This can occur in one of the many vessels used in propagation, conditioning or fermentation. This includes propagation tanks, conditioning tanks, starter tanks, fermentations tanks, piping and heat exchangers between these units.

Bacterial or microbial contamination reduces the fermentation product yield in three main ways. First, the sugars that could be available for yeast to produce alcohol are consumed by the bacteria or other undesirable microorganisms and diverted from alcohol production. In addition to reducing yield, the end products of bacterial metabolism, such as lactic acid and acetic acid, inhibit yeast growth and yeast fermentation/respiration, which results in less efficient yeast production. Finally, the bacteria or other undesirable microorganisms compete with the yeast for nutrients other than sugar.

After the fermentation stream or vessel has become contaminated with bacteria or other undesirable microorganisms, those bacteria or other microorganisms can grow much more rapidly than the desired yeast. The bacteria or other microorganisms compete with the yeast for fermentable sugars and retard the desired bio-chemical reaction resulting in a lower product yield. Bacteria also produce unwanted chemical by-products, which can cause spoilage of entire fermentation batches. Removing these bacteria or other undesirable microorganisms allows the yeast to thrive, which results in higher efficiency.

As little as a one percent decrease in ethanol yield is highly significant to the fuel ethanol industry. In larger facilities, such a decrease in efficiency will reduce income from 1 million to 3 million dollars per year.

Some previous methods of reducing bacteria or other undesirable microorganisms during propagation, conditioning and fermentation take advantage of the higher temperature and pH tolerance of yeast over other microorganisms. This is done by applying heat to or lowering the pH of the yeast solution. However, these processes are not entirely effective in retarding bacterial growth. Furthermore, the desirable yeast microorganisms, while surviving, are stressed and not as vigorous or healthy. Thus, the yeasts do not perform as well.

The predominant trend in the ethanol industry is to reduce the pH of the mash to less than 4.5 at the start of fermentation. Lowering the pH of the mash reduces the population of some species of bacteria. However it is much less effective in reducing problematic bacteria, such as lactic-acid producing bacteria, and is generally not effective for wild yeast and molds. It also significantly reduces ethanol yield by stressing the yeast.

Another approach involves washing the yeast with phosphoric acid. This method does not effectively kill bacteria and other microorganisms. It can also stress the yeast, thereby lowering their efficiency.

Yet another method is to use heat or harsh chemicals and sterilize process equipment between batches. However this method is only effective when equipment is not in use. It is ineffective at killing bacteria and other microorganisms within the yeast mixture during production.

In yet another method, antibiotics have previously been added to yeast propagation, conditioning or fermentation batch to neutralize bacteria. Fermentation industries typically apply antibiotics to conditioning, propagation and fermentation processes. Antibiotic dosage rates range between 0.1 to 3.0 mg/L and generally do not exceed 6 mg/L.

However, problems exist with using antibiotics in conditioning, propagation and fermentation. Antibiotics are expensive and can add greatly to the costs of large-scale production. Moreover, antibiotics are not effective against all strains of bacteria, such as antibiotic-resistant strains of bacteria. Overuse of antibiotics can lead to the creation of additional variants of antibiotic-resistant strains of bacteria.

Antibiotic residues and establishment of antibiotic-resistant strains is a global issue. These concerns may lead to future regulatory action against the use of antibiotics. One area of concern is distillers grain that is used for animal feed. European countries do not allow the byproducts of an ethanol plant to be sold as animal feed if antibiotics are used in the facility. Distiller grain sales account for up to 20% of an ethanol plant earnings. Antibiotic concentration in the byproduct can range from 1-3% by weight, thus negating this important source of income.

In addition, there are other issues to consider when using antibiotics. Calculating the correct dosage of antibiotic can be a daunting task. Even after dosages have been determined, mixtures of antibiotics should be constantly or at least frequently balanced and changed in order to avoid single uses that will lead to antibiotic-resistant strains. Sometimes the effective amount of antibiotic cannot be added to the fermentation mixture. For example, utilizing over 2 mg/L of Virginiamycin will suppress fermentation but over 25 mg/L is required to inhibit grown of Weisella confusa, an emerging problematic bacteria strain.

Fermentation plants can experience infections. This occurs when undesirable microorganism levels increase to above a normal or allowable level. This can occur due to process design, poor quality feed stock or other contributing factors. When this occurs antibiotic usage is usually increased to compensate for the infection. These conditions instigate overuse of antibiotic which can stress yeast and impact efficiency or cause regulatory non-compliance.

Stabilized sodium chlorite (SSC), produced from a 1 to 25% sodium chlorite solution has many industrial and municipal uses. When sodium chlorite reacts in an acidic environment it can form chlorine dioxide (ClO₂). When produced and handled properly, stabilized sodium chlorite is an effective and powerful biocide, disinfectant and oxidizer.

Stabilized sodium chlorite has been used as a disinfectant in the food and beverage industries, wastewater treatment, industrial water treatment, cleaning and disinfections of medical wastes, textile bleaching, odor control for the rendering industry, circuit board cleansing in the electronics industry, and uses in the oil and gas industry. It is an effective biocide at low concentrations and over a wide pH range. ClO₂ is desirable because when it reacts with an organism in water, it reduces to chlorite ion and then to chloride, which studies to date have shown does not pose a significant adverse risk to human health.

Recently, it was discovered that chlorine dioxide effectively reduces undesirable microorganisms during propagation, conditioning and/or fermentation while encouraging propagation and/or conditioning of the desirable microorganisms and increase their efficiency in fermentation. This is discussed in co-owned U.S. patent application Ser. No. 11/626,272, filed Jan. 23, 2007, entitled “Apparatus and Method for Treatment of Microorganisms During Propagation, Conditioning and Fermentation,” which claims priority benefits from U.S. Provisional Patent Application Ser. No. 60/775,615, filed Feb. 22, 2006, entitled “Apparatus and Method for Treatment of Yeast During Propagation, Conditioning and Fermentation.” Both of these applications are hereby incorporated by reference in their entirety.

Since as little as a one percent decrease in ethanol yield is highly significant to the fuel ethanol industry, ethanol producers are constantly looking for ways to increase efficiency.

Industries that employ fermentation for beverages have historically applied hops acid to propagation and fermentation to control unwanted microbiology that compete with the yeast for nutrients. With the recent expansion of fuel ethanol, hops acids have been utilized to a minor degree to address unwanted, gram positive microbiology. Competing microbiology to yeast results in an economic issue in fuel ethanol as unwanted microbiology, primarily Lactobacillus and Acetobacter, reduce the efficiency of fermentation. In beverage, competing microbiology to yeast not only reduce efficiency but can alter the aesthetics and taste of the final product.

Stabilized sodium chlorite is an intermediate product to forming chlorine dioxide that is a bactericide with a much greater degree of efficacy than hops acid. Hops acids utilized in the recommended dosage rates act as a bacteriastatic agent, effective against gram positive bacteria only—not a bactericide as chlorine dioxide does.

### SUMMARY OF THE INVENTION

In evaluation of stabilized sodium chlorite solution for use in conditioning, propagation and fermentation, it may be determined that not only is the solution compatible with hops acid but is synergistic when applying both technologies simultaneously. The combination of these products may provide a powerful, non antibiotic, antimicrobial treatment.

An embodiment of the current method for reducing undesirable microorganism concentration, promoting yeast propagation, and increasing yeast efficiency in an aqueous fluid stream comprises (a) introducing a quantity of fermentable carbohydrate to an aqueous fluid stream, (b) introducing a quantity of yeast to the aqueous fluid stream, (c) introducing an aqueous sodium chlorite solution into the aqueous fluid stream, and (d) introducing a hops acid extract stream into the aqueous fluid stream. These steps can be performed sequentially or in a different order.

In the foregoing method, the “undesirable” microorganisms intended to be reduced are those that compete for nutrients with the desirable microorganisms, such as yeast and Trichoderma that promote in the fermentation processes involved here. In this regard, the aqueous stabilized sodium chlorite and hops acid extract solution employed in the present method do not appear to detrimentally affect the growth and viability of desirable, fermentation-promoting microorganisms, but do appear to eliminate or at least suppress the growth of undesirable microorganisms that interfere with the fermentation process. Moreover, the elimination or suppression of undesirable microorganisms appears to have a favorable effect on the growth and viability of desirable microorganisms, for the reasons set forth in the Background section.

In one embodiment, the stabilized sodium chlorite solution has a concentration in the range of 10 to 200 mg/L as sodium chlorite to produce adequate chlorine dioxide utilizing the pH of the media in conjunction with the hops acid extract at a concentration of from about 0.1 to about 5 ppm.

The stabilized sodium chlorite solution produces chlorine dioxide, in a non-equipment basis, utilizing the acidity already present in the propagator and or fermentor. Examples of non-equipment based methods of ClO₂ generation include dry mix chlorine dioxide packets that include both a chlorite precursor packet and an acid activator packet and buffered sodium chlorite solutions ranging from 1 to 25%. In one embodiment, the ClO₂ solution is in the form of an aqueous solution having a concentration of less than about 15 mg/L and the hops acid extract has a concentration of from about 0.1 to about 5 ppm—the ClO₂ solution is produced from 10 to 200 mg/L of stabilized sodium chlorite solution addition. In another embodiment the stabilized sodium chlorite solution is in the form of an aqueous solution having a concentration of between about 10 and about 200 mg/L and the hops acid extract has a concentration of from about 0.1 to about 5 ppm.

An embodiment of the current apparatus for reducing undesirable microorganisms, promoting producing microorganism propagation, and increasing efficiency comprises a stabilized sodium chlorite batch tank, a hops acid extract tank and a process vessel for containing an aqueous microorganism solution. The stabilized sodium chlorite batch tank has an outlet for exhausting an aqueous stabilized sodium chlorite solution from the batch tank. The process vessel is fluidly connected to the batch tank. The process vessel is also fluidly connected to the hops acid extract tank. In operation, introducing the stabilized sodium chlorite and hops acid extract to the process vessel promotes propagation of producing microorganisms present in the vessel.

In one preferred embodiment, the batch tank is capable of exhausting an aqueous stabilized sodium chlorite solution that has a concentration of from 10 to 200 mg/L of stabilized sodium chlorite solution.

The process vessel can be a conditioning tank, heatable, capable of performing liquefaction or a yeast propagation vessel. The process vessel could also be a fermentation tank having an inlet for producing microorganisms, an inlet for water, an inlet for fermentation chemicals and an outlet for the fermentation product connecting to processing equipment.

Another embodiment of the current method for reducing undesirable microorganism concentration, promoting desirable microorganism propagation, and increasing desirable microorganism efficiency in an aqueous fluid stream comprises (a) introducing a quantity of cellulose to an aqueous fluid stream, (b) introducing a quantity of desirable microorganisms to the aqueous fluid stream, (c) introducing an aqueous stabilized sodium chlorite solution into the aqueous fluid stream and (d) introducing an aqueous hops acid extract stream into the aqueous fluid stream. These steps can be performed sequentially or in a different order

### DETAILED DESCRIPTION OF PREFERRED EMBODIMENT(S)

In the present method, the concentrations of bacteria and other undesirable microorganisms are reduced while simultaneously propagation and/or conditioning of desirable microorganisms is encouraged, and the efficiency of those desirable microorganisms in fermentation and an apparatus for carrying out this method increased.

Previously, chlorine dioxide produced from stabilized sodium chlorite was determined to be effective at reducing the concentration of bacteria and other undesirable microorganisms while simultaneously encouraging propagation and/or conditioning of desirable microorganisms and increasing the efficiency of those desirable microorganisms in fermentation. In evaluation of stabilized sodium chlorite for use in conditioning, propagation and fermentation, it may be determined that not only is stabilized sodium chlorite compatible with hops acid extract but is synergistic when applying both technologies simultaneously. Isomerized alpha extract is used as an example throughout this application. However, it is contemplated that other hops acid extracts could be used. For example beta acid compounds, alpha acids, isomerized alpha acids, rho isomerized alpha acids, tetra isomerized alpha acids, hexa isomerized alpha acids and hop leaf could be used.

Plant scale evaluations may determine that adding a small amount of hops acid extract, for example about 0.1 to about 5 ppm in addition to and simultaneously with stabilized sodium chlorite results in a synergistic effect. The addition of stabilized sodium chlorite and hops acid extract may simultaneously result in improved microbiology efficacy, enhanced ethanol production, reduced glycerol formation and increased yeast viability and propagation.

The production of fuel ethanol by yeast fermentation is used as an example. However, this is merely one illustration and should not be understood as a limitation. Other fermentation products could include distilled spirits, beer, wine, pharmaceuticals, pharmaceutical intermediates, baking products, nutraceuticals (foodstuff that provides health benefits, such as fortified foods and dietary supplements), nutraceutical intermediates and enzymes. The current method could also be utilized to treat yeast used in the baking industry. Other fermenting microorganisms could also be substituted such as the fungi and bacteria typically used in cellulosic ethanol production, Trichoderma reesei, Trichoderma viride, and Clostridium Ijungdahlii.

The fermentation process begins with the preparation of a fermentable carbohydrate. In ethanol production, corn is one possible fermentable carbohydrate. Other carbohydrates including cereal grains and cellulose-starch bearing materials, such as wheat or milo, could also be substituted. Cellulosic biomass such as straw and cornstalks could also be used. Cellulosic ethanol production has recently received attention because it uses readily available nonfood biomass to form a valuable fuel.

In corn-based ethanol production the corn is ground into a fine powder called meal. The meal is then mixed with water and enzymes, such as alpha-amylase, and passed through a cooker in order to liquefy the starch. A product known as corn mash results.

A secondary enzyme, such as glucoamylase, will also be added to the mash to convert the liquefied starch into a fermentable sugar. The glucoamylase cleaves single molecules of glucose from the short chain starches, or dextrins. The glucose molecules can then be converted into ethanol during fermentation.

Yeast, small microorganisms capable of fermentation, will also be added to the corn mash. Yeast are fungi that reproduce by budding or fission. One common type of yeast is Saccharomyces cerevisia, the species predominantly used in baking and fermentation. Non-Sacharomyces yeasts, also known as non-conventional yeasts, are naturally occurring yeasts that exhibit properties that differ from conventional yeasts. Non-conventional yeasts are utilized to make a number of commercial products such as amino acids, chemicals, enzymes, food ingredients, proteins, organic acids, nutraceuticals, pharmaceuticals, cosmetics, polyols, sweeteners and vitamins. Some examples of non-conventional yeasts include Kuyberomyces lactis, Yarrowia lipolytica, Hansenula polymorphs and Pichia pastoris. The current methods and apparatus are applicable to intermediates and products of both Sacharomyces and non-conventional yeast.

Most of the yeast used in fuel ethanol plants and other fermentation processes are purchased from manufacturers of specialty yeast. The yeast are manufactured through a propagation process and usually come in one of three forms: yeast slurry, compressed yeast or active dry yeast. Propagation involves growing a large quantity of yeast from a small lab culture of yeast. During propagation the yeast are provided with the oxygen, nitrogen, sugars, proteins, lipids and ions that are necessary or desirable for optimal growth through aerobic respiration.

Once at the distillery, the yeast may undergo conditioning. The objectives of both propagation and conditioning are to deliver a large volume of yeast to the fermentation tank with high viability, high budding and a low level of infection by other microorganisms. However, conditioning is unlike propagation in that it does not involve growing a large quantity from a small lab culture. During conditioning, conditions are provided to re-hydrate the yeast, bring them out of hibernation and allow for maximum anaerobic growth and reproduction.

Following propagation or conditioning, the yeast enter the fermentation process. The glucoamylase enzyme and yeast are often added into the fermentation tank through separate lines as the mash is filling the fermentation tank. This process is known as simultaneous saccharification and fermentation or SSF. The yeast produce energy by converting the sugars, such as glucose molecules, in the corn mash into carbon dioxide and ethanol.

The fermentation mash, now called “beer” is distilled. This process removes the 190 proof ethanol, a type of alcohol, from the solids, which are known as whole stillage. These solids are then centrifuged to get wet distillers grains and thin stillage. The distillers grains can be dried and are highly valued livestock feed ingredients known as dried distillers grains (DDGS). The thin stillage can be evaporated to leave a syrup. After distillation, the alcohol is passed through a dehydration system to remove remaining water. At this point the product is 200 proof ethanol. This ethanol is then denatured by adding a small amount of denaturant, such as gasoline, to make it unfit for human consumption.

The propagation, conditioning and fermentation processes can be carried out using batch and continuous methods. The batch process is used for small-scale production. Each batch is completed before a new one begins. The continuous fermentation method is used for large-scale production because it produces a continuous supply without restarting every time. The current method and apparatus are effective for both methods.

During the propagation, conditioning or fermentation process the mash or the fermentation mixture can become contaminated with other microorganisms, such as spoilage bacteria, wild yeast or killer yeast. These microorganisms compete with the yeast for fermentable sugars and retard the desired bio-chemical reaction resulting in a lower product yield. They can also produce unwanted chemical by-products, which can cause spoilage of entire fermentation batches. Wild yeast are a primary concern in the beverage industry because they can cause taste and odor problems with the final product. Killer yeast produce a toxin that is lethal to the desired alcohol producing yeast.

Producers of ethanol attempt to increase the amount of ethanol produced from one bushel of cereal grains, which weigh approximately 56 pounds (25.4 kilograms). Contamination by microorganisms lowers the efficiency of yeast making it difficult to attain or exceed the desired levels of 2.8-2.9 gallons per bushel (0.42-0.44 liters per kilogram). Reducing the concentration of microorganisms will encourage yeast propagation and/or conditioning and increase yeast efficiency making it possible to attain and exceed these desired levels.

Previously, it was determined that stabilized sodium chlorite (SSC) can be added at various points in the propagation, conditioning and/or fermentation processes to kill unwanted microorganisms and promote growth and survival of the desirable microorganisms. This stabilized sodium chlorite can be added as an aqueous solution. The stabilized sodium chlorite can be added during propagation, conditioning and/or fermentation. The stabilized sodium chlorite solution can be added to cook vessels, fermentation tanks, propagation tanks, conditioning tanks, The stabilized sodium chlorite solution can also be added to the interstage heat exchange system or heat exchangers.

Similarly, hops acid extract are useful for killing bacteria, wild yeasts, killer yeasts and molds while allowing yeast or other producing microorganisms to survive and thrive. Fermentation industries typically apply hops acid extracts to propagation and fermentation. Typically, hops acid extract dosage rates range between 15 and 50 ppm as active product when utilized independently.

In evaluation of stabilized sodium chlorite for use in conditioning, propagation and fermentation, it may be determined that not only is stabilized sodium chlorite compatible with hops acid extracts, such as isomerized alpha extract, but is synergistic when applying both technologies simultaneously. The stabilized sodium chlorite and hops acid extract may not compete or decrease the effectiveness of the other. Rather, applying stabilized sodium chlorite and hops acid extract may simultaneously increase ethanol yield over that achieved by adding either by itself.

Plant scale evaluations may determine that adding a small amount of hops acid extract, for example about 0.1 to about 5 ppm, in addition to and simultaneously with stabilized sodium chlorite results in a synergistic effect. The addition of stabilized sodium chlorite and hops acid extract may simultaneously result in improved microbiology efficacy, enhanced ethanol production, reduced glycerol formation and increased yeast viability and propagation.

Evaluations can be conducted at fuel ethanol facilities utilizing stabilized sodium chlorite and the hops acid extract. The facility can be dosed stabilized sodium chlorite to the propagator, fermentor and interstage heat exchanger at a dosage rate of between about 1 and about 50 mg/L. Hops acid extract (Iso-Alpha extract 30%) can be simultaneously applied to the propagator and fermentor at a rate of between about 0.1 and about 5 ppm.

Ethanol efficiency in the plant may be increased by addition of stabilized sodium chlorite and hops acid extract simultaneously. No detrimental or inhibitory effect may be noted between stabilized sodium chlorite and hops acid extract.

The hops acid extract can be added simultaneously with the stabilized sodium chlorite at the various points in the propagation, conditioning and/or fermentation processes where chorine dioxide solution was previously added. The hops acid extract can be added to cook vessels, fermentation tanks, propagation tanks, conditioning tanks, starter tanks or during liquefaction. The hops acid extract solution can also be added to the interstage heat exchange system or heat exchangers.

As mentioned above, stabilized sodium chlorite and hops acid extract can be added directly into the fermentation mixture. This can be done by adding the stabilized sodium chlorite and hops acid extract in conjunction with the yeast and glucoamylase, for example during the SSF stage. Stabilized sodium chlorite dosages from 10 to 200 mg/L with hops acid extract dosages of between 0.5 and 5 ppm can be added directly into the fermentation mixture.

Stabilized sodium chlorite and hops acid extract can also be added during propagation and/or conditioning. For example ClO₂ can be added to the yeast slurry before SSF replacing the acid washing step. Chlorine dioxide dosages of 10 to 200 mg/L used with hops acid extract dosages of between 0.5 and 6 ppm can be added during propagation and/or conditioning.

The stabilized sodium chlorite and hops acid extract solution is introduced at some point during the production of ethanol. The stabilized sodium chlorite and hops acid extract solution can be added during propagation, conditioning and/or fermentation. The stabilized sodium chlorite and hops acid extract solution can also be added directly to the corn mash. The stabilized sodium chlorite and hops acid extract solution can be added to cook vessels, fermentation tanks, propagation tanks, conditioning tanks, starter tanks or during liquefaction.

Stabilized sodium chlorite and hops acid extract can also be used simultaneously to achieve improved results in the production of cellulosic ethanol. Cellulosic ethanol is a type of ethanol that is produced from cellulose, as opposed to the sugars and starches used in producing carbohydrate based ethanol. Cellulose is present in non-traditional biomass sources such as switch grass, corn stover and forestry. This type of ethanol production is particularly attractive because of the large availability of cellulose sources. Cellulosic ethanol, by the very nature of the raw material, introduces higher levels of contaminants and competing microorganism into the fermentation process. Stabilized sodium chlorite and hops acid extract used simultaneously could be particularly helpful in cellulosic ethanol production as an antimicrobial agent.

There are two primary processes of producing alcohol from cellulose. One process is a hydrolysis process that utilizes a fungi such as Trichoderma reesei and Trichoderma viride. The other is a gasification process using a bacteria such as Clostridium Ijungdahlii. ClO₂ and hops acid extract could be utilized in either process.

In the hydrolysis process the cellulose chains are broken down into five carbon and six carbon sugars before the fermentation process. This is either done chemically and enzymatically.

In the chemical hydrolysis method the cellulose can be treated with dilute acid at high temperature and pressure or concentrated acid at lower temperature and atmospheric pressure. In the chemical hydrolysis process the cellulose reacts with the acid and water to form individual sugar molecules. These sugar molecules are then neutralized and yeast fermentation is used to produce ethanol. Stabilized sodium chlorite and hops acid extract could be used during the yeast fermentation portion of this method as outlined above.

Enzymatic hydrolysis can be carried out using two methods. The first is known as direct microbial conversion (DMC). This method uses a single microorganism to convert the cellulosic biomass to ethanol. The ethanol and required enzymes are produced by the same microorganism. Stabilized sodium chlorite and hops acid extract could be used during the propagation/conditioning or fermentation steps with this specialized organism.

The second method is known as the enzymatic hydrolysis method. In this method cellulose chains are broken down using cellulase enzymes. These enzymes are typically present in the stomachs of ruminants, such as cows and sheep, to break down the cellulose that they eat. In this process the cellulose is made via fermentation by cellulolytic fungi such as Trichoderma reesei and Trichoderma viride.

The enzymatic method is typically carried out in four or five stages. The cellulose is pretreated to make the raw material, such as wood or straw, more amenable to hydrolysis. Next the cellulase enzymes are used to break the cellulose molecules into fermentable sugars. Following hydrolysis, the sugars are separated from residual materials and added to the yeast. The hydrolyzate sugars are fermented to ethanol using yeast. Finally, the ethanol is recovered by distillation. Alternatively, the hydrolysis and fermentation can be carried out together by using special bacteria or fungi that accomplish both processes. When both steps are carried out together the process is called sequential hydrolysis and fermentation (SHF).

Stabilized sodium chlorite and hops acid extract can be introduced for microbiological efficacy at various points in the enzymatic method of hydrolysis. Stabilized sodium chlorite and hops acid extract could be used in the production, manufacture and fermentation of cellulase enzymes made by Trichoderma and other fungi strains. The stabilized sodium chlorite and hops acid extract can be added in the cellulosic simultaneous saccharification and fermentation phase (SSF). The stabilized sodium chlorite and hops acid extract can be introduced in the sequential hydrolysis and fermentation (SHF) phase. They could also be introduced at a point before, during or after the fermentation by cellulolytic fungi that create the cellulase enzymes. Alternatively the stabilized sodium chlorite and hops acid extract could be added during the yeast fermentation phase, as discussed above.

The gasification process does not break the cellulose chain into sugar molecules. First, the carbon in the cellulose is converted to carbon monoxide, carbon dioxide and hydrogen in a partial combustion reaction. Then, the carbon monoxide, carbon dioxide and hydrogen are fed into a special fermenter that uses a microorganism such as Clostridium Ijungdahlii that is capable of consuming the carbon monoxide, carbon dioxide and hydrogen to produce ethanol and water. Finally, the ethanol is separated from the water in a distillation step. Stabilized sodium chlorite and hops acid extract could be used as an antimicrobial agent in the fermentation step involving microorganisms such as Clostridium Ijungdahlii that are capable of consuming carbon monoxide, carbon dioxide and hydrogen to produce ethanol and water.

Another embodiment of the current technology is an apparatus for carrying out the fermentation process with an integrated stabilized sodium chlorite and hops acid extract system.

The apparatus comprises a batch tank that holds an aqueous stabilized sodium chlorite solution. The concentration of the stabilized sodium chlorite solution in the batch tank can vary across a wide range in order to create a stabilized sodium chlorite stream of the proper dosage. The stabilized sodium chlorite solution is then exhausted from the batch tank through an outlet at a specified dosage rate to create a solution of the desired concentration. In one embodiment the dosed stabilized sodium chlorite solution has a concentration of between about 10 and about 200 mg/L.

The apparatus has a hops acid extract tank. In the hops acid extract tank, hops acid extract, such as isomerized alpha extract, is dissolved in water to form a hops acid extract solution. The concentration of the hops acid extract solution in the batch tank can vary across a wide range. The hops acid extract solution is then exhausted from the batch tank through an outlet at a specified dosage rate to create a solution of the desired concentration. In one embodiment the dosed hops acid extract solution has a concentration between about 0.1 and 5 ppm. The hops acid extract tank is typically a pre-mix tank.

A process vessel containing an aqueous microorganism solution is fluidly connected to the batch tank and the hops acid extract tank via outlets on the batch tank and hops acid extract tank. The process vessel could be a cook vessel, fermentation tank, conditioning tank, starter tank, propagation tank, liquefaction vessel and/or piping or heat exchanger between these units. Introducing the stabilized sodium chlorite and hops acid extract solution into the process vessel is capable of promoting propagation of producing microorganism present while simultaneously decreasing the concentration of undesirable microorganisms.

While particular elements, embodiments and applications of the present invention have been shown and described, it will be understood, of course, that the invention is not limited thereto since modifications can be made by those skilled in the art without departing from the scope of the present disclosure, particularly in light of the foregoing teachings.

What is claimed is:

1. A method of reducing undesirable microorganism concentration, promoting yeast propagation/conditioning, and increasing yeast efficiency in an aqueous fluid stream employed in a fermentation process, the method comprising the steps of:
(a) introducing a quantity of fermentable carbohydrate to said stream;
(b) introducing a quantity of yeast to said stream;
(c) introducing an aqueous stabilized sodium chlorite solution into said stream; and
(d) introducing an aqueous hops acid extract stream into said stream.

2. The method of claim 1 wherein said steps are performed sequentially.

3. The method of claim 1 where stabilized sodium chlorite is added in the necessary quantity to produce the required mg/L of ClO₂O.

4. The method of claim 1 wherein said stabilized sodium chlorite solution has a concentration requirement to produce up to 15 mg/L of ClO₂O.

5. The method of claim 1 wherein said ClO₂ solution, from the stabilized sodium chlorite solution, has a concentration between about 5 and about 50 mg/L.

6. The method of claim 1 wherein said hops acid extract is isomerized alpha extract.

7. The method of claim where said aqueous hops acid extract stream has a dosage rate of about 0.1 to 5 ppm.

8. The method of claim 1 wherein said stabilized chlorine dioxide is an aqueous solution having a concentration of 20 to 200 mg/L to produce the necessary chlorine dioxide mg/L.

9. The method of claim 1 wherein said ClO₂ produced from stabilized sodium chlorite, is in the form of an aqueous solution having a concentration between about 10 and about 75 mg/L.

10. The method of claim 1 wherein said ClO₂ is produced by dry mix chlorine dioxide packets having a chlorite precursor packet and an acid activator packet and from a 1 to 25% buffered sodium chlorite solution.

11. An apparatus for reducing undesirable microorganism concentration, promoting producing organism propagation/conditioning, and increasing efficiency employed in a fermentation process, the apparatus comprising:
(a) a stabilized sodium chlorite batch tank, said stabilized sodium chlorite batch tank comprising an outlet for exhausting an aqueous stabilized sodium chlorite solution;
(b) a hops acid extract vessel for exhausting an aqueous hops acid extract stream from said hops acid extract vessel; and
(c) a process vessel for containing an aqueous microorganism solution, said process vessel fluidly connected to said stabilized sodium chlorite batch tank and said hops acid extract tank;
wherein introducing said stabilized sodium chlorite and hops acid extract solution from said stabilized sodium chlorite batch tank and said hops acid extract tank to said vessel promotes propagation of producing microorganisms present in said vessel.

12. The apparatus of claim 11 wherein said process vessel is heatable.

13. The apparatus of claim 11 wherein said process vessel is a fermentation tank having an inlet for producing microorganisms, an inlet for water, an inlet for fermentation chemicals and an outlet for the fermentation product connecting to processing equipment.

14. The apparatus of claim 11 wherein said process vessel is capable of performing liquefaction.

15. The apparatus of claim 11 wherein said process vessel is a yeast propagation tank.

16. The apparatus of claim 11 wherein said process vessel is a yeast conditioning tank.

17. The apparatus of claim 11 wherein said aqueous stabilized sodium chlorite solution exhausted from said stabilized sodium chlorite batch tank is dosed to a concentration between about 10 mg/L and about 200 mg/L.

18. The apparatus of claim 11 wherein said aqueous hops acid extract stream exhausted from said hops acid extract tank is dosed to a concentration of between about 0.1 and about 5 ppm.

19. A method of reducing undesirable microorganism concentration, promoting desirable microorganism propagation/conditioning, and increasing desirable microorganism efficiency in an aqueous fluid stream employed in a fermentation process, the method comprising the steps of:
(a) introducing a quantity of cellulose to said stream;
(b) introducing a quantity of desirable microorganisms to said stream;
(c) introducing an aqueous stabilized sodium chlorite solution into said stream; and
(d) introducing hops acid extract into said stream.

20. The method of claim 19 wherein said steps are performed sequentially.

21. A method of reducing residual byproduct of antibiotic in a fermentation process, the method comprising the steps of:
(a) introducing stabilized sodium chlorite into said fermentation process; and
(b) introducing hops acid extract into said fermentation process.

22. A method of reducing residual byproduct of chlorine dioxide in a fermentation process, the method comprising the steps of:
(a) introducing hops acid extract into said fermentation process;
(b) introducing a reduced amount of aqueous stabilized sodium chlorite solution into said fermentation process.
